(19) United States
(12) Patent Application Publication
(10) Pub. No.: US 2023/0273034 A1
(43) Pub. Date: Aug. 31, 2023

(54) INFORMATION PROCESSING APPARATUS AND NON-TRANSITORY COMPUTER READABLE MEDIUM STORING PROGRAM

(71) Applicant: Agama-X Co., Ltd., Tokyo (JP)

(72) Inventor: Kengo TOKUCHI, Kanagawa (JP)

(73) Assignee: Agama-X Co., Ltd., Tokyo (JP)

(21) Appl. No.: 18/142,583

(22) Filed: May 3, 2023

Related U.S. Application Data
(63) Continuation of application No. 16/742,924, filed on Jan. 15, 2020.

(30) Foreign Application Priority Data
Aug. 6, 2019 (JP) 2019-144786

(51) Int. Cl. G01C 21/36; G01C 21/34

(52) U.S. Cl. CPC G01C 21/3617; G01C 21/3664; G01C 21/367; G01C 21/3694; G01C 21/3697; G01C 21/3415; G01C 21/3623

(57) ABSTRACT

An information processing apparatus includes a processor configured to associate danger information with a location where biological information indicative of the danger information has been measured from a user.

### RELATED APPLICATIONS

This application is a Continuation of U.S. patent application Ser. No. 16/742,924 filed on Jan. 15, 2020, which claims the benefit of priority of Japanese Patent Application No. 2019-144786 filed Aug. 6, 2019 the contents of which are incorporated herein by reference in their entirety.

### FIELD AND BACKGROUND OF THE INVENTION

### (i) Technical Field

The present invention relates to an information processing apparatus and a non-transitory computer readable medium storing a program.

### (ii) Related Art

JP2014-134515A describes a technique in which an SNS server transmits post information and guidance information to a smartphone, the smartphone displays the post information, sets one of the displayed post information as a destination, and guides a route to a destination.

JP2003-194568A describes a technique in which on a portion corresponding to a congestion section of a route graphic representing a guidance route, information section graphic is displayed so as to be visually recognized such that the display color of the route graphic is changed only in that part.

### SUMMARY OF THE INVENTION

Aspects of non-limiting embodiments of the present disclosure relate to an information processing apparatus and a non-transitory computer readable medium storing a program, capable of informing a user of a danger which the user hardly notices.

Aspects of certain non-limiting embodiments of the present disclosure address the above advantages and/or other advantages not described above. However, aspects of the non-limiting embodiments are not required to address the advantages described above, and aspects of the non-limiting embodiments of the present disclosure may not address advantages described above.

According to an aspect of the present disclosure, there is provided an information processing apparatus including a processor configured to associate danger information with a location where biological information indicative of the danger information has been measured from a user.

### BRIEF DESCRIPTION OF THE SEVERAL VIEWS OF THE DRAWINGS

Exemplary embodiment(s) of the present invention will be described in detail based on the following figures, wherein:

FIG. 1 is a block diagram illustrating a configuration of an information processing system according to the present exemplary embodiment;

FIG. 2 is a diagram illustrating a management table;

FIG. 3 is a diagram illustrating a management table;

FIG. 4 is a diagram illustrating a management table;

FIG. 5 is a diagram schematically showing an area where there are buildings, roads, and the like;

FIG. 6 is a diagram illustrating an image representing a map;

FIG. 7 is a diagram illustrating a screen;

FIG. 8 is a diagram illustrating a screen;

FIG. 9 is a diagram schematically showing an area where there are buildings, roads, and the like;

FIG. 10 is a diagram illustrating an image representing a map;

FIG. 11 is a diagram schematically showing an area where there are buildings, roads, and the like;

FIG. 12 is a diagram illustrating an image representing a map;

FIG. 13 is a diagram showing a list of routes;

FIG. 14 is a diagram schematically showing an area where there are buildings, roads, and the like;

FIG. 15 is a diagram schematically showing an area where there are buildings, roads, and the like;

FIG. 16 is a diagram illustrating an image representing a map; FIG. 17 is a diagram schematically showing an area where there are buildings, roads, and the like; and

FIG. 18 is a diagram illustrating an image representing a map.

### DESCRIPTION OF SPECIFIC EMBODIMENTS OF THE INVENTION

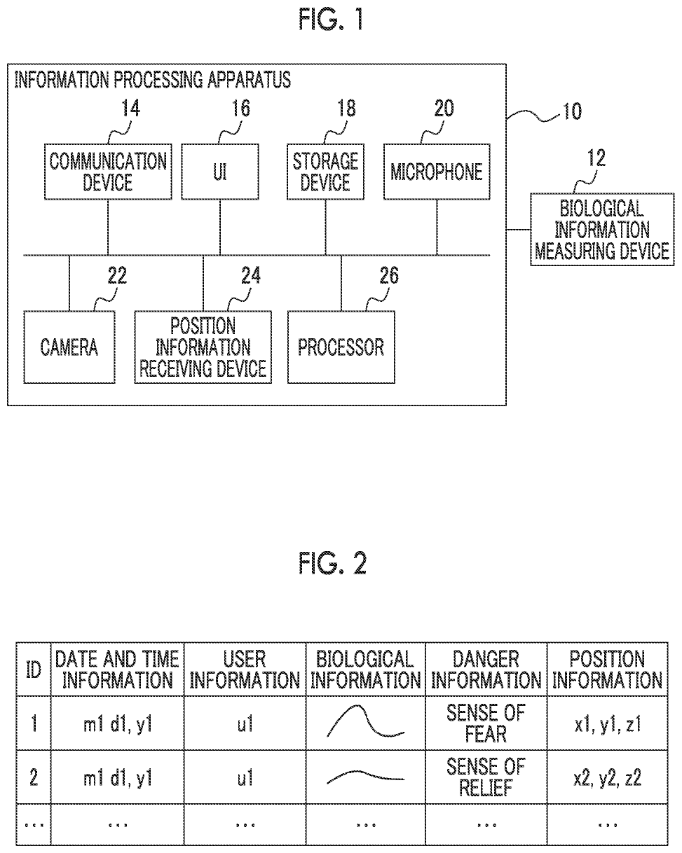

An information processing system according to the present exemplary embodiment will be described with reference to FIG. 1. FIG. 1 illustrates an example of the hardware configuration of the information processing system according to the present exemplary embodiment.

The information processing system according to the present exemplary embodiment includes an information processing apparatus 10 and one or a plurality of biological information measuring devices. In the example shown in FIG. 1, the information processing system includes three biological information measuring devices. Specifically, the information processing system includes biological information measuring devices 12A, 12B, and 12C. Hereinafter, in a case where it is not necessary to distinguish between the biological information measuring devices 12A, 12B, and 12C, these are referred to as “biological information measuring device 12”. The configuration illustrated in FIG. 1 is merely an example, and the number of biological information measuring devices 12 included in the information processing system may be one or may be four or more. In addition, the information processing system according to the present exemplary embodiment may include other devices (for example, an external device such as a server) other than these devices.

The information processing apparatus 10 and the biological information measuring device 12 are configured to communicate with each other. The communication may be wired communication using a cable, or wireless communication. That is, the information processing apparatus 10 and the biological information measuring device 12 may transmit and receive information to and from each other, by being physically connected to each other by a cable, or may transmit and receive information to and from each other by wireless communication. The biological information measuring devices 12 may also communicate with each other by wired communication or wireless communication. For example, near field communication, Wi-Fi (registered trademark), or the like is used as the wireless communication. Wireless communication of standards other than these may be used. Near field communication is, for example, Bluetooth (registered trademark), radio frequency identifier (RFID), NFC, or the like. The information processing apparatus and the biological information measuring device 12 may communicate with each other through a communication path such as a local area network (LAN) or the Internet. The information processing apparatus 10 and the biological information measuring device 12 may communicate with other devices by wired communication or wireless communication.

The information processing apparatus 10 is, for example, a personal computer (hereinafter referred to as “PC”), a tablet PC, a smartphone, a mobile phone, or other devices. The information processing apparatus 10 may be a terminal device (for example, a tablet PC, a smartphone, a mobile phone, or the like) that can be carried by the user, or may be a device that is installed on a table or the like and used.

The biological information measuring device 12 includes a sensor, an electrode, and the like, and is configured to measure a user's biological information. Each biological information measuring device 12 is configured to measure different types of biological information, for example. Of course, a part or all of the biological information measuring devices 12 may be configured to measure the same type of biological information. Each biological information measuring device 12 may be configured to measure one type of biological information or may be configured to measure a plurality of types of biological information.

The biological information measuring device 12 transmits the biological information measured from the own device to the information processing apparatus 10. The biological information measuring device 12 may transmit the biological information to the information processing apparatus 10 every time the biological information is measured, or store the biological information and transmit the biological information to the information processing apparatus 10 at predetermined time intervals, or transmit the biological information to the information processing apparatus 10 at a timing designated by the user. The biological information measuring device 12 may receive the biological information measured by another type of biological information measuring device 12 from the other biological information measuring device 12, and transmit the biological information measured from the own device and the biological information measured from the other type of biological information measuring device 12 to the information processing apparatus 10.

The biological information measuring device 12 may analyze the biological information measured from the own device or the other type of biological information measuring device, and transmit information indicating the analysis result to the information processing apparatus 10. For example, the biological information measuring device 12 may include a processor, and the processor may analyze the biological information. Of course, the analysis may be performed by the information processing apparatus 10.

In addition, the biological information measuring device 12 includes a battery, and may be driven by power supplied from the battery, or may be driven by receiving power supplied from the information processing apparatus 10. Further, the biological information measuring device 12 may include a storage device, a communication device, and the like.

The biological information measuring device 12 may be a wearable device that measures biological information by the entire biological information measuring device 12 being worn on a user. For example, the biological information measuring device 12 may be a device worn on the user's head, a bearable device worn on the user's ear, or a device worn on the user's arm, hand, wrist, or finger (for example, a wristwatch-type device), a device worn around the user's neck, or a device worn on the user's body or legs.

The biological information is various types of physiological information and anatomical information emitted from a user who is a living body. The category of the concept of biological information includes, for example, a brain wave, a pulse rate, a blood pressure, a heart rate, an electrocardiogram waveform, an electromyogram waveform, an eye movement, and a subject's movement. These are only examples of such biological information, and other types of physiological information or anatomical information may be used as the biological information. The biological information measuring device 12 may measure one piece of information among these pieces of biological information, or may measure a plurality of pieces of information. For example, the biological information measuring device 12A measures the user's brain waves, the biological information measuring device 12B measures the user's pulse rate, and the biological information measuring device 12B measures the user's myoelectric waveform. This is only an example, and biological information measuring devices 12 may measure other types of biological information, and one biological information measuring device 12 may measure a plurality of types of biological information.

The information processing apparatus 10 receives biological information from the biological information measuring device 12, and analyzes the biological information, stores the biological information, outputs the biological information, stores information indicating the analysis result of the biological information, and outputs information indicating the analysis result of the biological information. Of course, analysis of biological information may be performed by the biological information measuring device 12. Outputting the biological information includes, for example, displaying the biological information, outputting the biological information as voice information, and the like. Outputting information indicating the analysis result of the biological information includes, for example, displaying information indicating the analysis result, outputting the analysis result as voice information, and the like. The information processing apparatus 10 may transmit biological information and information indicating the analysis result to another apparatus.

The information processing apparatus 10 may include one or a plurality of biological information measuring devices 12. That is, one or a plurality of biological information measuring devices 12 may be incorporated in the information processing apparatus 10. For example, the information processing apparatus may include at least one of the biological information measuring device 12A, 12B, or 12C. For example, all of the biological information measuring devices 12A, 12B, and 12C may be incorporated in the information processing apparatus 10 to constitute one device. The entire information processing apparatus 10 including the biological information measuring devices 12A, 12B, and 12C may be worn by the user to measure biological information. That is, the information processing apparatus 10 may be a wearable apparatus. For example, the information processing apparatus 10 may be a device worn on the user's head, a bearable device worn on the user's ear, or a device worn on the user's arm, hand, wrist, or finger (for example, a wristwatch-type device), a device worn around the user's neck, or a device worn on the user's body or legs.

Of course, the information processing apparatus 10 and the biological information measuring device 12 may be separate devices. For example, the information processing apparatus 10 may be a smartphone, and the biological information measuring device 12 may be a wearable device worn by a user.

Hereinafter, the configuration of the information processing apparatus 10 will be described in detail.

The information processing apparatus 10 includes, for example, a communication device 14, a UI 16, a storage device 18, a microphone 20, a camera 22, a position information receiving device 24, and a processor 26. The information processing apparatus 10 may include other configurations.

The communication device 14 is a communication interface, and has a function of transmitting data to other apparatuses and a function of receiving data transmitted from other apparatuses. The communication device 14 may have a wireless communication function or may have a wired communication function. The communication device 14 may communicate with other devices by using, for example, near field communication, or may communicate with other devices through a communication path such as a LAN or the Internet. The communication device 14 communicates with the biological information measuring device 12, and receives the biological information transmitted from the biological information measuring device 12. The communication device 14 may transmit control information for controlling the operation of the biological information measuring device 12 to the biological information measuring device 12.

The UI 16 is a user interface, and includes a display device and an operation device. The display device is a liquid crystal display, an EL display, or the like. The operation device is a keyboard, input keys, an operation panel, or the like. The UI 16 may be a UI such as a touch panel that has both a display device and an operation device. In addition, a microphone 20 to be described later may be included in the UI 16, and a speaker that emits sound may be included in the UI 16.

The storage device 18 is a device that constitutes one or a plurality of storage areas for storing various types of data. The storage device 18 is, for example, a hard disk drive, various memories (for example, RAM, DRAM, ROM, or the like), other storage devices (for example, an optical disk), or a combination thereof. One or a plurality of storage devices 18 are included in the information processing apparatus 10.

The microphone 20 is a device that collects sound waves. For example, the voice of the user of the information processing apparatus 10, sounds around the information processing apparatus 10, and the like are input to the microphone 20, and sound data is generated by the microphone 20. The sound represented by the sound data generated by the microphone 20 corresponds to an example of environment information indicating the environment around the information processing apparatus 10.

The camera 22 is a capturing device. For example, the surroundings of the information processing apparatus 10 is captured by the camera 22 and image data representing the surroundings is generated. The image data may be moving image data or still image data. An image represented by image data captured by the camera 22 corresponds to an example of environment information indicating an environment around the information processing apparatus 10.

The position information receiving device 24 is a device configured to receive position information of the information processing apparatus 10. The position information receiving device 24 is configured to receive position information of the information processing apparatus 10 by using, for example, global positioning system (GPS). The position information is, for example, information indicating the latitude and longitude of the information processing apparatus 10, coordinate information indicating the position of the information processing apparatus 10 in a predetermined coordinate system, and the like. Further, the position information may include information indicating the height. The position information receiving device 24 may receive the position information of the information processing apparatus 10 by using a technique other than GPS.

The processor 26 is configured to associate the danger information based on the biological information of the user with the position information indicating the position where the biological information is obtained. The biological information is measured by the biological information measuring device 12. The position information is received by the position information receiving device 24. The danger information is generated by analyzing the biological information. The analysis may be performed by the processor 26, may be performed by the biological information measuring device 12 that measures the biological information, or may be performed by another biological information measuring device 12 different from the biological information measuring device 12 that has measured the biological information, or may be performed by another device such as a server. In the following, it is assumed that the processor 26 analyzes biological information.

The danger information is information indicating that the user whose biological information is measured feels danger, or that the user has a feeling similar to danger. The feelings that are similar to danger are, for example, a sense of anxiety, fear, pressure, discomfort, and the like. Whether or not the user feels danger and whether or not the user has a feeling similar to danger are specified by analyzing the biological information. For example, in a case where specific biological information reflecting danger or feeling similar thereto is measured, or in a case where a change amount of certain biological information is equal to or greater than a threshold, the processor 26 determines whether or not the user feels danger or has a feeling similar to danger. Specific biological information reflecting danger or feeling similar thereto is determined in advance. Of course, the processor 26 may determine that the user feels a danger or has a feeling similar to a danger by using a known technique.

In a case where a plurality of different types of biological information are measured, the processor 26 determines whether or not the user feels danger and whether or not the user has a feeling similar to danger, based on the plurality of types of biological information. For example, in a case where a plurality of types of specific biological information reflecting danger or feeling similar thereto is measured, or in a case where the change amount of the plurality of types of biological information is equal to or greater than a change amount threshold, the processor 26 may determine whether or not the user feels danger or has a feeling similar to danger.

In a case where one or a plurality of pieces of biological information representing danger information are measured and another one or a plurality of pieces of biological information that does not represent danger information are measured, the processor 26 may determine whether or not the user feels danger or has a feeling similar to danger, based on the magnitude relationship between the number of pieces of biological information representing danger information and the number of pieces of biological information that does not represent danger information. For example, in a case where the number of pieces of biological information representing danger information is larger than the number of pieces of biological information that does not represent danger information, the processor 26 may determine that the user feels danger or has a feeling similar to danger.

As another example, the processor 26 may attach a score corresponding to the importance of the biological information to each pieces of biological information, and determine whether or not the user feels danger or has a feeling similar to danger, based on the magnitude relationship between the total value of the score of the biological information representing the danger information and the total value of the score of the biological information that does not represent the danger information. For example, since a brain wave is biological information that is important in determining whether or not the user feels danger, a higher score is given than other types of biological information is given thereto. In a case where the total value of the score of the biological information representing the danger information is larger than the total value of the score of the biological information that does not represent the danger information, the processor 26 determines that the user feels danger or has a feeling similar to danger.

Further, in a case where specific biological information reflecting relief, relaxation, or feeling similar thereto is measured, or in a case where the amount of change in certain type of biological information is less than a change amount threshold, the processor 26 may determine whether or not the user feels relief or is relaxed. That is, in this case, the processor 26 may determine that the user does not feel danger and does not have a feeling similar to danger.

For example, the processor 26 may determine whether or not the user feels danger and whether or not the user has a feeling similar to danger, based on a brain wave that is an example of biological information.

For example, γ waves (for example, brain waves of 30 Hz or higher) may be measured when the user feels anxiety or when the user is excited. In a case where γ waves are measured, the processor 26 may determine that the user feels a danger or the user has a feeling similar to a danger. In a case where the length of the period in which the γ waves are continuously measured is equal to or greater than the length threshold, the processor 26 may determine that the user feels a danger or the user has a feeling similar to a danger.

Further, β waves (for example, brain waves of 13 to 30 Hz) may be measured when the user is slightly nervous. In a case where β waves are measured, the processor 26 may determine that the user feels a little dangerous or that the user has a little feeling that is similar to danger. In a case where the length of the period in which the β waves are continuously measured is equal to or greater than the length threshold, the processor 26 may determine that the user feels a little dangerous or that the user has a little feeling that is similar to danger. In a case where the γ waves and the β waves are measured alternately, and the length of the period in which the γ waves and the β waves are measured is equal to or greater than the length threshold, the processor 26 may determine that the user feels a danger or the user has a feeling similar to a danger.

Further, α waves (for example, brain waves of 7 to 13 Hz) may be measured when the user is relaxed. In a case where a waves are measured, the processor 26 may determine that the user is relaxed. That is, the processor 26 may determine that the user does not feel danger and the user does not have a feeling similar to danger. In a case where the length of the period in which the α waves are continuously measured is equal to or greater than the length threshold, the processor 26 may determine that the user is relaxed.

In a case where the ratio of γ waves measured during a predetermined unit period is equal to or greater than the threshold of the ratio, the processor 26 may determine that the user feels a danger or the user has a feeling similar to a danger.

The processor 26 may determine that the user feels a danger or the user has a feeling similar to a danger, based on the ratio of each brain wave measured during a predetermined unit period. For example, in a case where the ratio of γ waves measured during a unit period among the α wave, β wave, and γ wave is the highest, the processor 26 may determine that the user feels dangerous or the user has a feeling similar to a danger. In a case where the ratio of 13 waves measured during a unit period among the α wave, β wave, and γ wave is the highest, the processor 26 may determine that the user feels a little dangerous or that the user has a little feeling that is similar to danger. In a case where the ratio of α waves measured during a unit period among the α wave, β wave, and γ wave is the highest, the processor 26 may determine that the user does not feel danger and the user does not have a feeling that is similar to danger.

The processor 26 may determine whether the user feels danger or whether the user has a feeling similar to danger, based on the transition of the measured brain waves. Of course, the processor 26 may determine whether the user feels danger or whether the user has a feeling similar to danger, based on the brain waves, by using a known technique.

As another example, the processor 26 may determine whether or not the user feels danger or whether or not the user has a feeling similar to danger, based on the pulse rate that is an example of biological information. For example, in a case where the pulse rate is equal to or greater than the pulse rate threshold, the processor 26 may determine that the user feels a danger or the user has a feeling similar to a danger. In a case where the length of the period in which the pulse rates equal to or greater than the threshold are continuously measured is equal to or greater than the length threshold, the processor 26 may determine that the user feels a danger or the user has a feeling similar to a danger.

As another example, the processor 26 may determine whether or not the user feels danger or whether or not the user has a feeling similar to danger, based on the blood pressure that is an example of biological information. For example, in a case where the blood pressure is equal to or greater than the blood pressure threshold, the processor 26 may determine that the user feels a danger or the user has a feeling similar to a danger. In a case where the length of the period in which the blood pressure equal to or greater than the threshold is continuously measured is equal to or greater than the length threshold, the processor 26 may determine that the user feels a danger or the user has a feeling similar to a danger.

As another example, the processor 26 may determine whether or not the user feels danger or whether or not the user has a feeling similar to danger, based on the heart rate that is an example of biological information. For example, in a case where the heart rate is equal to or greater than the heart rate threshold, the processor 26 may determine that the user feels a danger or the user has a feeling similar to a danger. In a case where the length of the period in which the heart rates equal to or greater than the threshold are continuously measured is equal to or greater than the length threshold, the processor 26 may determine that the user feels a danger or the user has a feeling similar to a danger. A pulse rate may be used instead of or together with the heart rate.

As described above, the processor 26 determines whether the user feels danger or whether the user has a feeling similar to danger, based on the biological information. In a case where the user feels dangerous or in a case where the user has a feeling similar to danger, the processor 26 may generate danger information indicating that the user feels danger or the user has a feeling similar to danger, and associates the danger information with the position information indicating the position where the biological information representing the danger information is measured. The processor 26 records a position where the biological information representing the danger information is measured. Specifically, the processor 26 stores the danger information and the position information in the storage device 18 in association with each other. For example, the processor 26 stores management information including the danger information and the position information which are associated with each other, in the storage device 18. The management information may be stored in the storage device 18 or may be stored in another device such as a server without being stored in the storage device 18.

The processor 26 may further store, in the storage device 18, biological information representing danger information, date and time information indicating the date and time when the biological information is measured, user information for identifying the user whose biological information is measured, environment information indicating an environment around a position where the biological information representing danger information is measured, information indicating means of transportation of the user when the biological information representing danger information is measured, or the like in association with the danger information and the position information. The user information may include attribute information indicating user attributes. User attributes include, for example, the user's gender, age, physical characteristics (for example, height and weight), and mental characteristics (for example, fear).

In a case where the user does not feel danger, in a case where the user does not have a feeling similar to danger, in a case where the user feels relief, or in a case where the user is relaxed, the processor 26 may generate safety information indicating that the user does not feel danger, and record the safety information and the position information indicating a position where the biological information representing the safety information is measured in the management information in association with each other. Similarly to the danger information, the processor 26 may further record, in the management information, biological information representing safety information, date and time information indicating the date and time when the biological information is measured, user information for identifying the user whose biological information is measured, environment information indicating an environment around a position where the biological information representing safety information is measured, information indicating means of transportation of the user when the biological information representing safety information is measured, or the like in association with the safety information and the position information.

The processor 26 may determine whether or not the user feels danger or whether or not the user has a feeling similar to danger, by analyzing the biological information using artificial intelligence (that is, AI). Known artificial intelligence may be used, or in a case where artificial intelligence that determines a user's feeling based on one or a plurality of pieces of biological information is developed, the artificial intelligence may be used.

The processor 26 may provide various types of information to the user, by using the danger information or safety information associated with the position information. For example, the processor 26 may display a map on the display unit of the UI 16, and display danger information and safety information on the map. The processor 26 may display a route traveled by the user whose biological information is measured on the map. Further, in a case of guiding the route from the departure place to the destination, the processor 26 may display information on the position where the biological information representing the danger information is measured on the display unit of the UI 16, or may emit information on the position from a speaker as voice information. The same applies to safety information. These processes will be described in detail later.

Hereinafter, the information processing system according to the present exemplary embodiment will be described in more detail.

FIG. 2 shows a management table corresponding to an example of management information. The data of the management table may be stored in the storage device 18 or may be stored in another device such as a server.

In the management table, for example, an ID, date and time information, user information, biological information, danger information, and position information are associated with each danger information. The ID is information for managing each piece of information recorded in the management table. The date and time information is information indicating the date and time when the biological information associated with the date and time information is measured. The user information is information for identifying the user whose biological information associated with the user information is measured, and is, for example, a user ID, a user name, or a user account. The user information may include user attribute information. The biological information is information measured by the biological information measuring device 12. One or more pieces of biological information may be associated with one piece of danger information. As described above, the danger information is generated by analyzing the biological information associated with the danger information. The position information is information indicating the position where the biological information associated with the position information is measured, and is, for example, information indicating coordinate information or an address. In a case where biological information representing safety information is measured instead of biological information representing danger information, the safety information may be recorded in the management table.

For example, user information for identifying a user who has logged in to the information processing apparatus 10 is recorded in the management table. User information for identifying a user who uses the biological information measuring device 12 may be recorded in the management table. For example, in a case where a user who uses the biological information measuring device 12 is registered in the biological information measuring device 12 and a user who uses the biological information measuring device 12 is selected, user information for identifying the selected user may be recorded in the management table.

The biological information associated with the ID “1” is associated with danger information representing a sense of fear. That is, the user u1 feels fear at the position where the biological information is measured, that is, the position indicated by the position information associated with the biological information. For example, in a case where biological information representing a sense of fear is measured, the processor 26 records the date and time information indicating the date and time when the biological information is measured, user information, the biological information, danger information indicating that the user u1 feels fear, and the position information indicating the position where the biological information is measured, in the management table in association with each other. The same applies to information other than ID “1”.

Further, the biological information associated with the ID “2” is associated with safety information indicating a sense of relief. That is, the user u1 feels relief at the position where the biological information is measured. In this case, the biological information representing the danger information is not measured, but the biological information representing the safety information is measured, so the safety information is recorded in the management table.

By referring to the management table shown in FIG. 2, it is possible to specify where the user feels fear or relief at which position.

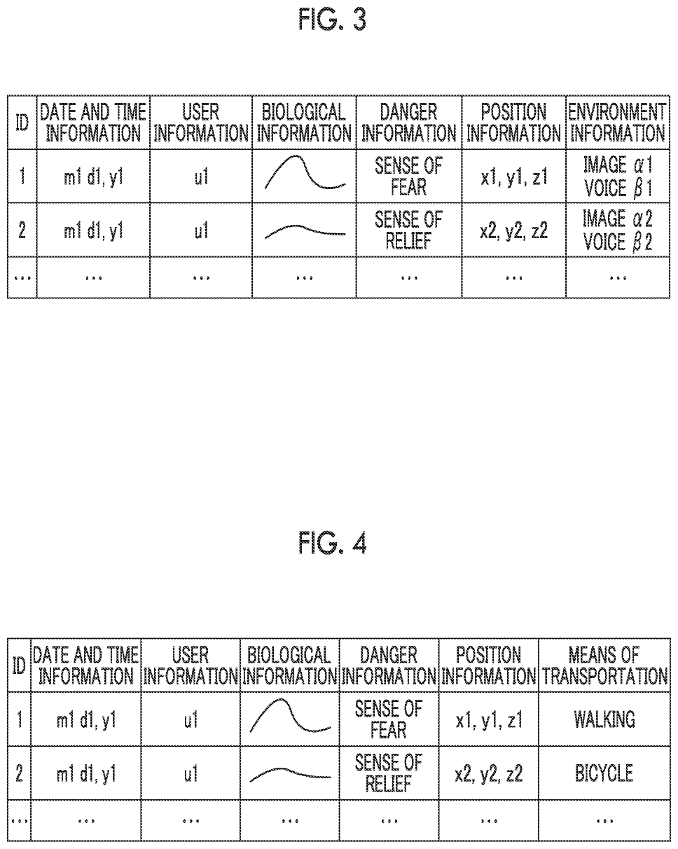

FIG. 3 shows another management table. In this management table, for example, an ID, date and time information, user information, biological information, danger information, position information, and environment information are associated with each danger information. The environment information is information indicating the environment around the information processing apparatus 10 when the biological information associated with the environment information is measured. For example, voice data obtained by the microphone 20 and image data generated by capturing by the camera 22 are recorded in the management table as an example of environment information.

For example, image data α1 and voice data β1 are associated with the biological information with ID “1”. The image data α1 is image data captured at the position and date and time when the user u1 feels fear, and is image data representing the environment around the information processing apparatus 10. The image data α1 may be image data captured during a period including the time when the user u1 feels fear and times before and after that time, or image data captured after the user u1 feels fear. The voice data β1 is voice data obtained at the position and date and time when the user u1 feels fear, and is data representing the sound around the information processing apparatus 10. The voice data β1 may be voice data obtained during a period including the time when the user u1 feels fear and times before and after that time, or voice data obtained after the user u1 feels fear. For example, ambient noise (for example, car sounds, human conversations, or the like), voice of the user u1, and the like are included in the voice data. For example, in a case where biological information representing a sense of fear is measured, the processor 26 records information indicating the date and time information indicating the date and time when the biological information is measured, user information, the biological information, danger information indicating that the user u1 feels fear, the position information indicating the position where the biological information is measured, and the environment information obtained at the position and date and time when the biological information is measured, in the management table in association with each other. The same applies to information other than ID “1”.

Further, the biological information associated with the ID “2” is associated with relief information indicating a sense of relief. In this case as well, the environment information is recorded in the management table in the same manner as the danger information. Note that the relief information is different from the danger information, but for convenience of explanation, the relief information is shown in the column of danger information in FIG. 2. The same applies to FIGS. 3 and 4.

By referring to the management table shown in FIG. 3, it is possible to specify where the user feels fear or relief, and further, it is possible to specify the ambient environment when the user feels fear or relief.

FIG. 4 shows another management table. In this management table, for example, an ID, date and time information, user information, biological information, danger information, position information, and information indicating means of transportation are associated with each danger information. The information indicating means of transportation is information indicating means of transportation of the user when the biological information associated with the information indicating means of transportation is measured. Means of transportation is, for example, walking, a bicycle, a car, a train, an airplane, or a ship. Information indicating more specific contents of each means of transportation may be included in the information indicating means of transportation. In a case where the user is traveling on a private car, a bus, a taxi, or the like, information indicating the private car, the bus, or the tax may be included in the information indicating means of transportation. The same applies to other types of means of transportation.

The user may designate the user's own means of transportation, using the UI 16. In this case, the processor 26 records information indicating means of transportation designated by the user in the management table in association with the biological information or the like. As another example, the processor 26 may estimate means of transportation of the user. For example, in a case where the user is moving with the information processing apparatus 10, the processor 26 measures the moving speed of the information processing apparatus 10 using an acceleration sensor or the like installed in the information processing apparatus 10, and estimates means of transportation of the user, based on the measured moving speed.

For example, the biological information with ID “1” is associated with information indicating walking as means of transportation. That is, the user u1 feels fear while walking, at the position indicated by the position information at the date and time indicated by the date and time information. In this way, by referring to the management table shown in FIG. 4, it is possible to specify where the user feels fear or relief, and further, it is possible to specify means of transportation of the user when the user feels fear or relief.

Further, the biological information associated with the ID “2” is associated with relief information indicating a sense of relief. In this case as well, the information indicating means of transportation is recorded in the management table in the same manner as the danger information.

The management tables shown in FIGS. 2 to 4 are only examples. The information indicating means of transportation shown in FIG. 4 may be further recorded in the management table shown in FIG. 3. User information include, for example, information indicating the user's gender, age, physical characteristics (for example, height and weight), mental characteristics (for example, fear), or the like.

For example, by recording also the user's gender in the management table, it is possible to specify from the management table, when and where what feeling what type of gender of the user has.

Hereinafter, the process by the information processing apparatus 10 according to the present exemplary embodiment will be described with a specific example.

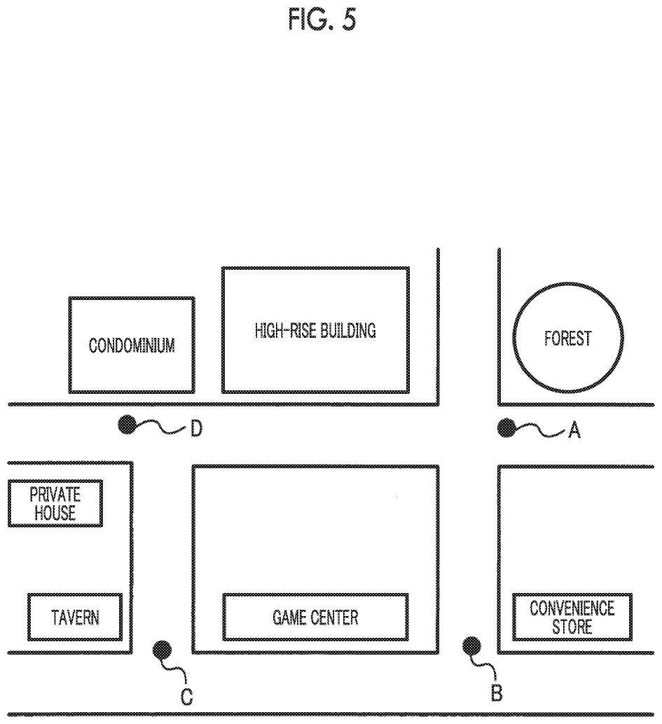

FIG. 5 schematically shows an area of a certain town. In this area, for example, buildings are built and roads are installed.

Since a point A is a point along the main street, but there are forests and a high-rise building around the point A, some people walking may feel a sense of pressure. There is no traffic light at point A.

There are a game center and a convenience store around the point B, and a relatively large number of young people visit the game center and the convenience store. There is a traffic light at point B, and even in a case where the traffic light is yellow, there are people who cross the road, so the driver of the car may feel fear.

There are a game center and a tavern around the point C, and there are many drunk people in the night time zone, so the point C is less secure than other places. Therefore, some people feel fear in the night time zone. There is a traffic light at point C.

There are a condominium and a high-rise building around point D. Because the balcony of the condominium faces the road, passersby sometimes feel gaze and uncomfortable. For example, women may be uncomfortable.

For example, the information processing apparatus 10 may transmit and receive various data by communicating with a base station (for example, a 5G base station) installed in a traffic light.

For example, the user u1 is moving while carrying the information processing apparatus 10 in a state where the user has logged in to the information processing apparatus 10. The user u1 feels pressure when walking through the point A, and in a case where the biological information representing the sense of pressure is measured by the biological information measuring device 12, the processor 26 records danger information indicating the sense of pressure and the position information indicating the point A in the management table in association with each other. Further, the processor 26 may record the date and time information indicating the date and time when the biological information representing the sense of pressure is measured, the user information of the user u1, and the biological information in association with the danger information and the position information in the management table. Further, the processor 26 may record the environment information indicating the environment around the point A in the management table in association with the danger information and the position information, or may record information indicating means of transportation of the user u1 in the management table in association with the danger information and the position information. The environment information is, for example, image data representing the surroundings of the point A and voice data obtained at the point A. The image data represents, for example, a high-rise building, a forest, a traffic situation, and the like. The voice data includes, for example, car noise.

Further, the user u1 feels fear when the user u1 drives a car and passes through the point B, and in a case where the biological information representing a sense of fear is measured by the biological information measuring device 12, the processor 26 records danger information indicating a sense of fear and the position information indicating the point B in the management table in association with each other. Similarly to the point A, the processor 26 may record date and time information, user information, environment information, and information indicating means of transportation in the management table in association with the danger information and the position information.

Further, the user u1 feels fear when walking through the point C, and in a case where the biological information representing a sense of fear is measured by the biological information measuring device 12, the processor 26 records danger information indicating a sense of fear and the position information indicating the point C in the management table in association with each other. Similarly to the point A, the processor 26 may record date and time information, user information, environment information, and information indicating means of transportation in the management table in association with the danger information and the position information.

Further, the user u1 feels uncomfortable when walking through the point D, and in a case where the biological information representing the discomfort is measured by the biological information measuring device 12, the processor 26 records danger information indicating the discomfort and the position information indicating the point D in the management table in association with each other. Similarly to the point A, the processor 26 may record date and time information, user information, environment information, and information indicating means of transportation in the management table in association with the danger information and the position information.

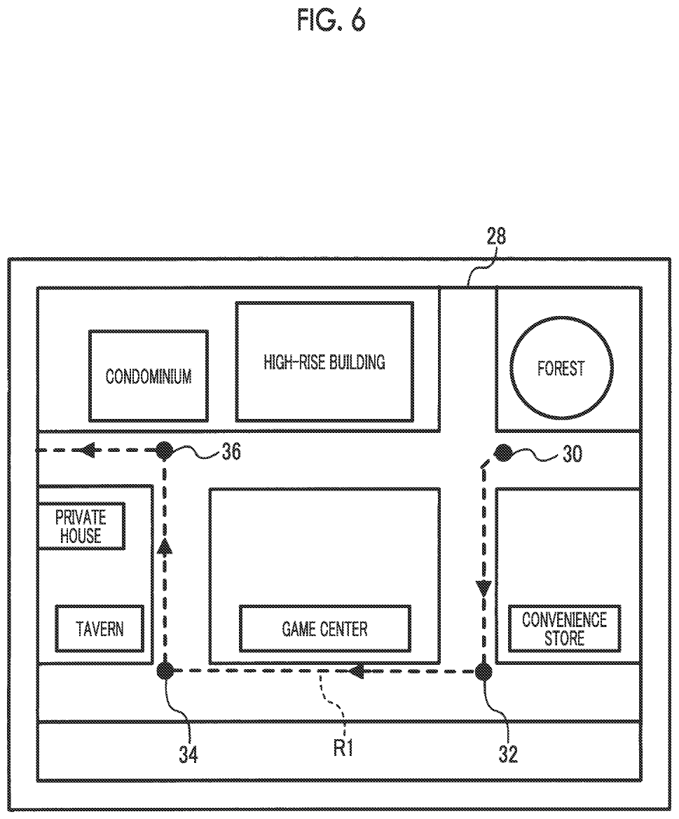

The processor 26 may display danger information recorded in the past on a map. For example, in a case where the user instructs to start the map application by using the UI 16, the processor 26 displays an image representing the map (hereinafter referred to as “map image”) on the display unit of the UI 16. FIG. 6 shows a map image. A map image 28 is displayed on the display unit of the UI 16. The processor 26 may display a map image representing a map of the area including the current position of the user on the display unit of the UI 16, or display a map image representing a map of the area designated by the user on the display unit of the UI 16. The map image 28 may be an image representing a map of an area including the current position of the user or an image representing a map of an area designated by the user.

The map image 28 is an image representing a map of the area shown in FIG. 5. The processor 26 displays danger information on the map image 28, based on the information recorded in the management table.

Only the danger information about the user using the information processing apparatus 10 may be displayed on the map image 28, or both the danger information about the user using the information processing apparatus 10 and the danger information about the other users may be displayed on the map image 28. The user who uses the information processing apparatus 10 is, for example, a user who has logged in to the information processing apparatus 10. Specifically, in a case where the user u1 has logged in to the information processing apparatus 10, only the danger information about the user u1 may be displayed on the map image 28, or the danger information about users other than the user u1 may also be displayed on the map image 28. Here, it is assumed that only danger information about the user u1 is displayed on the map image 28.

For example, in a case where biological information representing a sense of pressure has been measured at the point A in the past, a marker 30 indicating that biological information representing danger information has been measured is displayed at a position corresponding to the point A, on the map image 28.

In a case where biological information representing a sense of fear has been measured at a point B in the past, a marker 32 indicating that biological information representing danger information has been measured is displayed at a position corresponding to the point B, on the map image 28.

In a case where biological information representing a sense of fear has been measured at a point C in the past, a marker 34 indicating that biological information representing danger information has been measured is displayed at a position corresponding to the point C, on the map image 28.

In a case where biological information representing a sense of discomfort has been measured at a point D in the past, a marker 36 indicating that biological information representing danger information has been measured is displayed at a position corresponding to the point D, on the map image 28.

By referring to the map image 28, the user can recognize the position where the biological information representing the danger information has been measured in the past.

The processor 26 may display date and time information indicating the date and time when the biological information representing the danger information is measured, on the map image 28. The processor 26 displays, for example, date and time information indicating the date and time when the biological information representing the sense of pressure is measured at the point A in the vicinity of the marker 30. The same applies to other points. In a case where the user designates the marker on the map image 28, the processor 26 may display date and time information indicating the date and time when the biological information representing the sense of pressure is measured at the point A.

The processor 26 may display image data representing the environment around the position where the biological information representing the danger information is measured, on the map image 28. The processor 26 displays, for example, a surrounding image captured when the biological information representing the sense of pressure is measured at the point A, in the vicinity of the marker 30. The same applies to other points. In a case where the user designates the marker 30 on the map image 28, the processor 26 may display a surrounding image captured when the biological information representing the sense of pressure is measured at the point A.

The processor 26 may emit from a speaker, a sound around a position where the biological information representing the danger information is measured. For example, in a case where the user designates the marker 30 on the map image 28, the processor 26 emits from a speaker, ambient sound obtained when the biological information representing the sense of pressure is measured at the point A. The same applies to other points.

The processor 26 may display, on the map image 28, information indicating means of transportation of the user when the biological information representing the danger information is measured. The processor 26 displays, for example, information indicating means of transportation of the user u1 when the biological information representing the sense of pressure is measured at the point A, in the vicinity of the marker 30. The same applies to other points. In a case where the user u1 designates the marker 30 on the map image 28, the processor 26 may display information indicating means of transportation of the user when the biological information representing the sense of pressure is measured at the point A.

Further, the processor 26 may display a route traveled by the user, on the map image 28. For example, it is assumed that the user u1 moves in the order of points A, B, C, and D. The processor 26 stores the position information of each position received by the position information receiving device 24 in the storage device 18 as a history, and displays the route R1 traveled by the user on the map image 28, based on the position information of each position. In FIG. 6, the route R1 is indicated by a broken line. In this way, the user can recognize the position where the user has felt danger or the like on the route traveled by the user.

In a case where the user is at a position where biological information representing danger information has been measured in the past or is within a predetermined warning area including the position, the processor 26 may output a warning. For example, the processor 26 may display warning information indicating that biological information representing danger information has been measured in the past on the display unit of the UI 16, may emit a warning sound from a speaker, or may vibrate the information processing apparatus 10. When the map image 28 is displayed, in a case where the user is at a position where biological information representing danger information has been measured in the past or is within the warning area, the processor 26 may output a warning. Of course, even in a case where the map image 28 is not displayed, the processor 26 may output a warning.

For example, in a case where the user is at the point A or within a warning area including the point A, the processor 26 outputs a warning. The same applies to points B, C, and D.

The processor 26 may automatically record the danger information without receiving an instruction from the user, or may record the danger information when receiving an instruction from the user.

In the case where danger information is recorded when a user instruction is received, in a case where the biological information representing the danger information is measured, the processor 26 may notify the user that the biological information representing the danger information has been measured. The processor 26 may display information indicating that biological information representing danger information has been measured on the display unit of the UI 16, may emit a sound representing that the biological information representing the danger information has been measured from a speaker, or may vibrate the information processing apparatus 10.

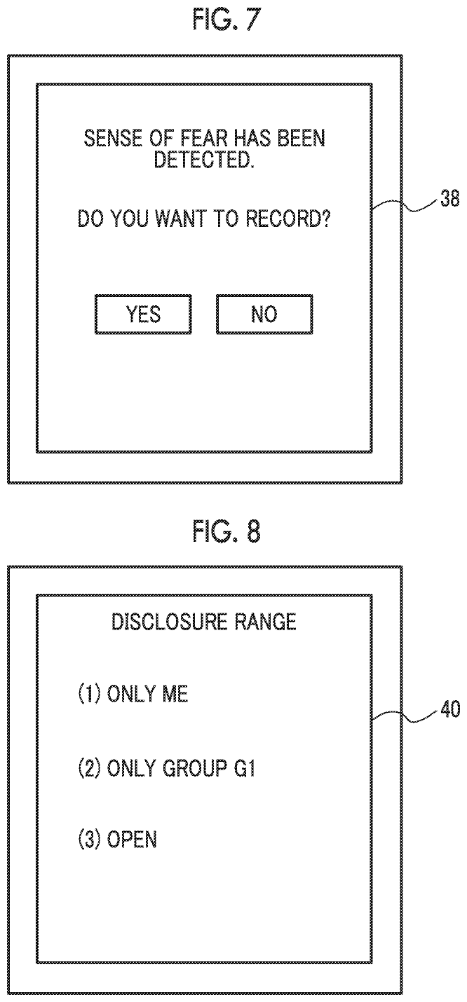

For example, as illustrated in FIG. 7, the processor 26 may display a message indicating that biological information representing danger information has been measured on the display unit of the UI 16. A screen 38 is displayed on the display unit of the UI 16, and a message indicating that biological information representing danger information has been measured (for example, a message indicating that a sense of fear has been detected) is displayed on the screen 38. Further, a message inquiring the user whether to record danger information is displayed on the screen 38. In a case where recording is instructed by the user on the screen 38 (for example, in a case where a “Yes” button is pressed), or recording is instructed by voice, the processor 26 records the danger information and the position information in the management table in association with each other. As described with reference to FIGS. 2 to 4, other types of information such as date and time information may be recorded in the management table. In a case where on the screen 38, the user instructs not to record the biological information (for example, when a “No” button is pressed), or in a case where the user instructs not to record the biological information by voice, the processor 26 does not record danger information.

Further, the processor 26 may share the danger information with other users. Other users are users other than the user whose biological information representing the danger information is measured. Other users are designated by, for example, a user whose biological information representing danger information is measured. Other users may be designated in advance, or may be designated when biological information representing danger information is measured. Sharing danger information with other users means performing processing such that other users can recognize the danger information. For example, in a case where biological information representing danger information is measured, the processor 26 may transmit the danger information and the position information to the addresses of other users by e-mail, or may allow other users to browse by a social media or social networking service (SNS). In a case where the danger information and the position information are recorded in an external device such as a server, the processor 26 may allow other users to access the danger information and the position information recorded in the external device. Further, the shared danger information may be displayed on the map image. For example, danger information about other users other than the user u1 may be displayed on the map image 28 shown in FIG. 6. The processor 26 may also share other information (for example, date and time information) recorded in the management table with other users.

In a case where danger information is recorded, the range of users who can refer to the danger information may be set. Here, the range of users who can refer to the danger information is referred to as a “disclosure range”. It can be said that the disclosure range is a range of users who share danger information.

FIG. 8 shows a screen 40 for setting the disclosure range. For example, in a case where the user instructs to record biological information, the processor 26 displays the screen 40 on the display unit of the UI 16. A list of disclosure ranges is displayed on the screen 40. For example, (1) “Only me”, (2) “Only the set range of users (for example, only group G1)”, and (3) “Open” are included in the list of disclosure ranges.
(1) “Only me” means that only the user whose biological information representing the danger information is measured can refer to the danger information.
(2) “Only the set range of users (for example, only group G1)” means that users belonging to the set range of users (for example, users belonging to the group G1) can refer to the danger information. The user range may be set in advance or may be set on the screen 40.
(3) “Open” means that there is no limit to the range of users who can refer to the danger information. That is, anyone can refer to the danger information.

In a case where (2) “Only the set range of users and (3) “Open” are set, the danger information is shared with other users.

Hereinafter, various examples will be described.

### EXAMPLE 1

In Example 1, the processor 26 guides a route from the departure place to the destination. That is, the processor 26 executes navigation. The departure place and the destination are designated by the user, for example. The departure place may be the user's current location. In a case where the user is at a position where biological information representing danger information has been measured in the past or is within a warning area including the position, the processor 26 outputs a warning. The processor 26 may output a warning using the danger information about the user to be guided, or may output a warning using the warning information about the user to be guided and the danger information about other users. For example, in a case where the danger information is shared, the processor 26 may output a warning by using the shared danger information. The user to be guided is a user who uses the navigation function, for example, a user who has logged in to the information processing apparatus 10.

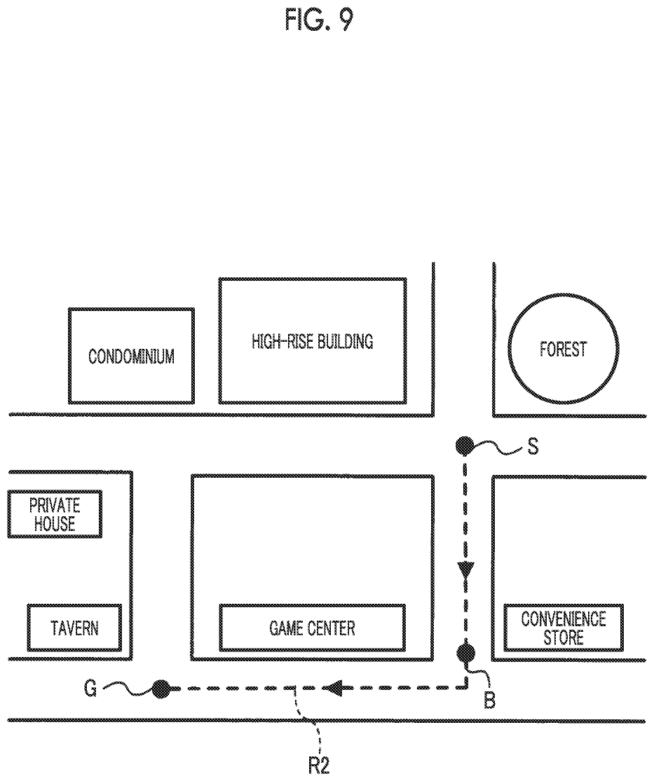

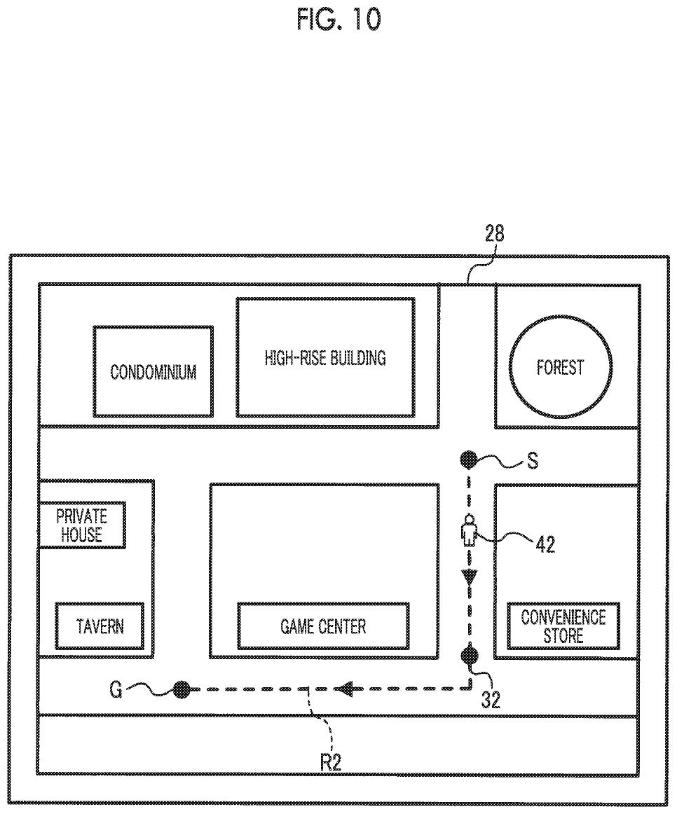

Hereinafter, Example 1 will be described in detail with reference to FIGS. 9 and 10. FIG. 9 schematically shows a certain area as in FIG. 5. FIG. 10 shows an image representing a map. Here, as an example, it is assumed that the user u1 uses the navigation function. The user u1 moves while carrying the information processing apparatus 10 in a state where the user has logged in to the information processing apparatus 10.

As shown in FIG. 9, for example, in a case where the departure place S and the destination G are set, the processor 26 searches for a route from the departure place S to the destination G. For example, a known technique is used for the route search. The processor 26 may search for a route for each means of transportation. For example, the processor 26 searches for routes for walking, private cars, buses, trains, and the like. The processor 26 may search for a plurality of routes having different distances, or may search for a plurality of routes having different times required for movement from the departure place S to the destination G, and may search for a plurality of routes having different costs required for the movement. In a case where a plurality of routes are searched, the processor 26 may display a list of the plurality of routes on the display unit of the UI 16. In a case where a route is selected by the user from the list, the processor 26 guides the route selected by the user. Here, as an example, a route R2 passing through the point B is set, and the processor 26 guides the route R2. The point B is a point where biological information representing a sense of fear is measured from the user u1.

In a case where the route guidance starts, as shown in FIG. 10, the processor 26 displays a map image 28 on the display unit of the UI 16. For example, the processor 26 displays the route R2 and the user image 42 indicating the current location of the user u1 on the map image 28.

In a case where the user u1 is at the point B or within a warning area including the point B, the processor 26 outputs a warning. The processor 26 may display on the map image 28 a message indicating that the biological information representing the danger information is measured at the point B, may emit a warning sound from a speaker, or may vibrate the information processing apparatus 10. Further, in a case where the user u1 is at the point B or in the warning area including the point B, the processor 26 may display the marker 32 on the map image 28. Even in a case where the user u1 is not in the warning area including the point B, the processor 26 may display the marker 32 on the map image 28.

Further, in a case where a difference between the current time when the user u1 is at the point B or the current time when the user u1 is within the warning area including the point B and the time when the biological information representing the danger information has been measured at the point B in the past is less than the threshold, the processor 26 may output a warning.

Further, in a case when the user is moving by the user's means of transportation when the biological information representing the danger information is measured, in a case where the user is at the position where the biological information representing the danger information is measured, or is within the warning area including the position, the processor 26 may output a warning.

For example, the point B is a point where biological information representing fear is measured when the user u1 is driving a car. Therefore, in a case where the user u1 is driving a bicycle, in a case where the user u1 is at the point B or within the warning area including the point B, the processor 26 outputs a warning. In a case where the user u1 is moving by another means of transportation other than the car, the processor 26 does not output a warning, even in a case where the user u1 is at the point B or within the warning area including the point B.

Further, in a case where the danger information is shared, the processor 26 may output a warning by using the danger information about other users than the user u1. For example, in a case where there is a point where biological information representing danger information is measured from another user other than the point B on the route R2, in a case where the user u1 is at the point or is within a warning area including the point, the processor 26 outputs a warning.

Further, the processor 26 may change the warning output mode, according to the attribute of the user who uses the navigation function.

For example, the processor 26 changes the warning output mode, according to whether the user is male or female. In a case where the user is female, the processor 26 outputs the warning more conspicuously than in a case where the user is male. To output a warning more conspicuously means to display a message with a larger warning text, to produce a larger warning sound, or the like.

As another example, the processor 26 may change the warning output mode, according to whether the user is afraid or not. In a case where the user is afraid, the processor 26 outputs the warning more conspicuously than in a case where the user is not afraid.

### EXAMPLE 2

In Example 2, in a case of guiding a route from the departure place to the destination, the processor 26 guides the route that avoids the dangerous area. The dangerous area is a position where the biological information representing the danger information described above is measured, or a predetermined warning area including the position. As another example, the dangerous area may be determined without being based on biological information. For example, the dangerous area may be determined by an administrative organization or a private organization. An area where an incident, an accident, a disaster, or the like has occurred may be determined as a dangerous area. In this case, the processor 26 acquires information on the dangerous area from a device used in an administrative organization or a private organization through a communication path. The information related to the dangerous area includes, for example, position information indicating the position of the dangerous area, information indicating the contents of the incident or accident occurring in the dangerous area, information indicating the date and time when the incident or accident occurs, and the like. The dangerous area may include a position where the biological information representing the danger information described above is measured, or a warning area including the position, and an area determined without being based on biological information.

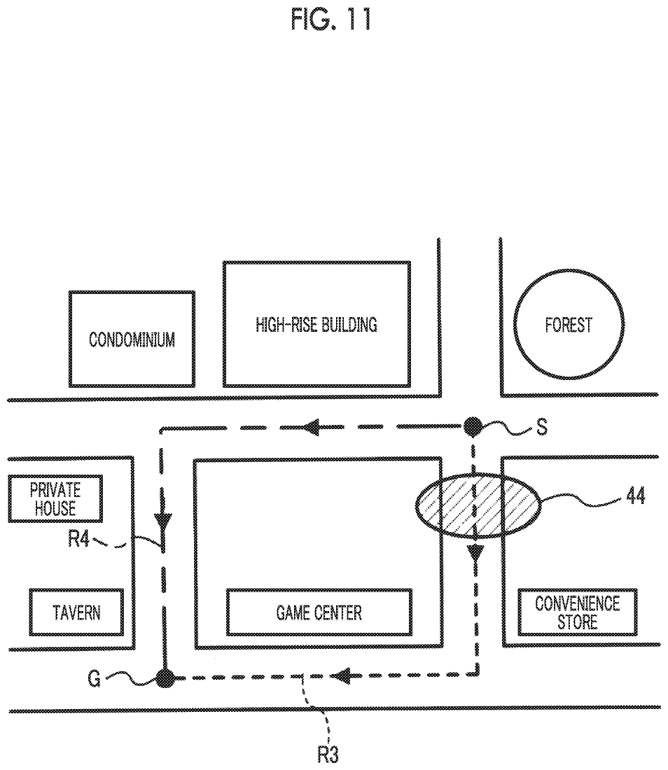

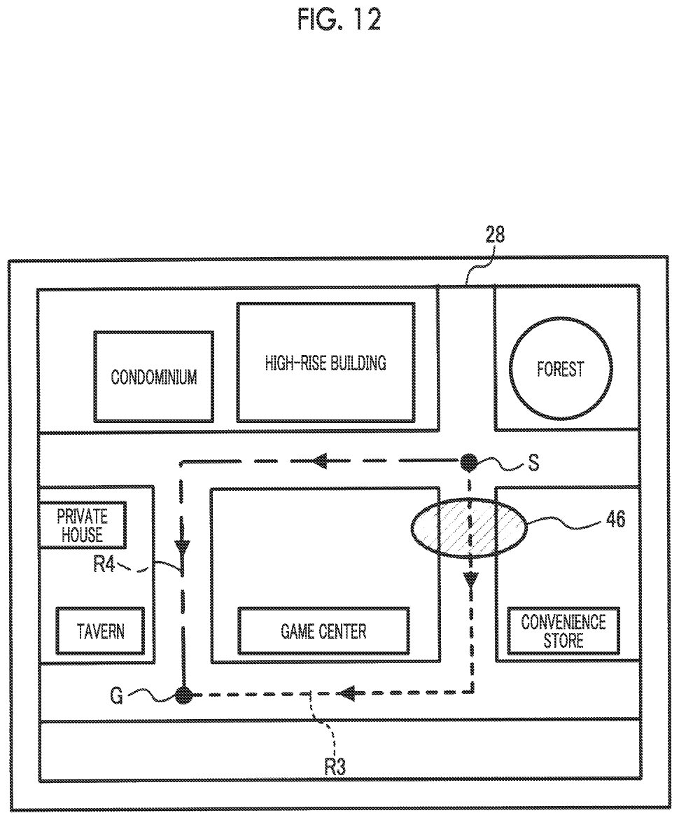

For example, as shown in FIG. 11, a departure place S and a destination G are set, and routes R3, R4 from the departure place S to the destination G are searched. The route R3 is a route that passes through the dangerous area 44. In this case, the processor 26 does not guide the route R3 and guides the route R4 that does not pass through the dangerous area 44. As shown in FIG. 12, the map image 28 is displayed on the display unit of the UI 16, and the processor 26 guides the route R4. The processor 26 may display an image 46 representing the dangerous area 44 on the map image 28.

Further, the processor 26 may display information indicating means of transportation for avoiding a danger generated from the dangerous area on the display unit of the UI 16, or may emit a sound representing means of transportation from a speaker. For example, in a case where a traffic accident or a disaster occurs on the ground, the processor 26 selects the subway as means of transportation for avoiding danger, and displays information indicating the subway on the display unit of the UI 16 or emits sound representing the subway from the speaker. Further, in a case where walking is dangerous, the processor 26 selects means of transportation other than walking as means of transportation for avoiding the danger.

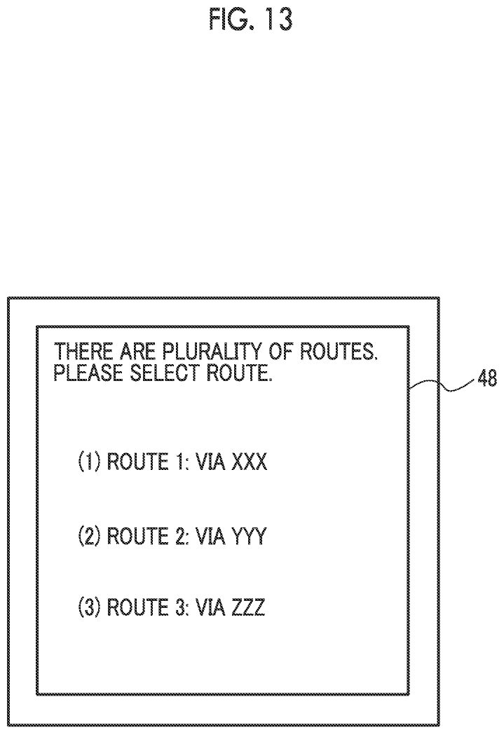

In a case where a plurality of routes avoiding the dangerous area are searched, the processor 26 may prioritize each route and display each route on the display unit of the UI 16. FIG. 13 shows the display example. A screen 48 representing a list of routes is displayed on the display unit of the UI 16. For example, routes 1, 2, and 3 are searched. The route 1 is the route with the highest priority, the route 2 is the route with the second highest priority, and the route 3 is the route with the third highest priority.

For example, the processor 26 determines the priority, based on the danger level of the dangerous area. For example, a danger level is determined in advance for each type of incident or each type of accident. The more violent incident and malicious the incident, the higher the danger level. For example, the danger level of a burglar case is higher than the danger level of a theft. Further, the greater the accident scale, the higher the danger level. For example, the danger level of an accident in which a casualty has occurred is higher than the danger level of an accident in which no casualty has occurred. Further, the greater the number of casualties, the higher the danger level. The processor 26 determines the danger level of the dangerous area, based on the information indicating the contents of the incident or accident that has occurred in the dangerous area, which is included in the information on the dangerous area, and determines the priority based on the danger level. The route passing through the dangerous area with a higher danger level has a lower priority. Further, the higher the frequency of incidents and accidents (for example, the number of incidents and accidents that has occurred during a predetermined unit period), the higher the danger level. Further, the danger level may be lowered with time. For example, the danger level decreases step by step with time. That is, the danger level becomes higher as the time when an incident or accident occurs is closer to the current time, and the danger level becomes lower as the time is farther from the current time.

For example, the route 1 is a route that does not pass through the dangerous area, the route 2 is a route that passes through the dangerous area with a low danger level, and the route 3 is a route that passes through the dangerous area with a high danger level.

In a case where the user selects a route on the screen 48, the processor 26 guides the route selected by the user.

Further, the processor 26 may determine the priority, based on the danger level, the required time, and the cost.

In addition, elements to be regarded as important may be set among a plurality of elements (for example, a danger level, required time, and cost) for determining priorities. The setting may be performed by the user or may be automatically set by the processor 26. For example, in a case where the user sets the cost as the element to be regarded as important, the processor 26 gives higher priority to a route with a lower cost.

Further, the processor 26 may share a route that avoids the dangerous area with a plurality of other users. Other users are users other than the user who sets the route, and are designated by the user who sets the route. Other users may be designated in advance, or may be designated when the route is shared with other users. For example, the processor 26 may transmit information indicating the route to the addresses of other users by e-mail, or may make other users browse the route by a social media or social networking service (SNS). In a case where the information indicating the route is stored in an external device such as a server, the processor 26 may allow other users to access the information indicating the route stored in the external device.

In a case where there are a plurality of routes that avoid the dangerous area, the processor 26 may share the plurality of routes with a plurality of other users. In this case, the processor 26 determines a route to be guided, based on an agreement among a plurality of users sharing the plurality of routes, from the plurality of routes. Then, the processor 26 guides the determined route. For example, each user who shares the plurality of routes designates a route to be guided using his or her terminal device (for example, a PC or a smartphone). Further, the user whose route is to be guided also designates the route to be guided using his or her information processing apparatus 10. Note that the number of routes that can be designated by the user may be one, or may be two or more. The number may be designated by the user whose route is to be guided. In a case where a route is designated by another user, information indicating the route designated by the other user is transmitted to the information processing apparatus 10 from the terminal device of the other user. For example, the processor 26 may determine the route with the highest number of designated routes as the routes to be guided, or determine one or a plurality of routes having the designated number equal to or greater than the threshold as the routes to be guided, or determine a route designated by all users as a route to be guided. In a case where a plurality of routes are determined as routes to be guided, a user whose route is guided designates a route to be finally guided from among the plurality of routes. A weight may be set for each user, and the weight may be reflected in a designated number. Further, in a case where another user (such as an administrator) who has an authority to set a route designates a route to be guided, the processor 26 may guide the route.

The dangerous area may be updated at a predetermined time interval or may be updated according to a user's instruction.

### EXAMPLE 3

In Example 3, in a case where the user is in the dangerous area, the processor 26 outputs a warning. The meaning of the dangerous area according to Example 3 is identical to the dangerous area according to Example 2. For example, in a case where the user is at a position where biological information representing danger information is measured or is within a warning area including the position, the processor 26 outputs a warning. For example, the processor 26 may display a message indicating that the user is in the dangerous area on the display unit of the UI 16, may emit a sound representing that the user is in the dangerous area from a speaker, or may vibrate the information processing apparatus 10.

Further, in a case where the user approaches the dangerous area, the processor 26 may output a warning. The case where the user approaches the dangerous area is a case where the distance between the user position and the dangerous area position is less than the distance threshold. The position of the dangerous area is the position of the end of the dangerous area closest to the user position, the center of the dangerous area, or the center of gravity of the dangerous area.

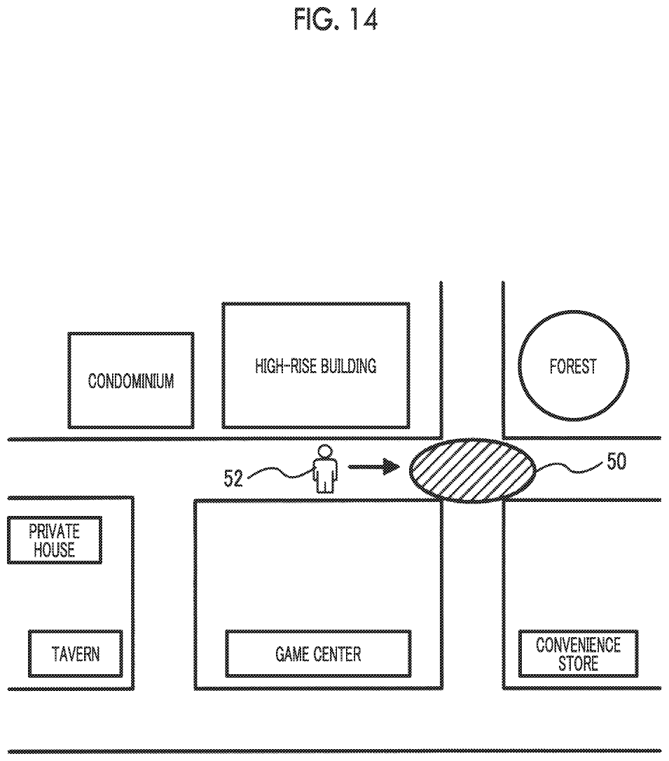

With reference to FIG. 14, Example 3 will be described with a specific example. For example, a dangerous area 50 is determined. In a case where the user 52 carrying the information processing apparatus 10 approaches the dangerous area 50, the processor 26 outputs a warning.

In a case where the user 52 moves away from the dangerous area 50, the processor 26 stops outputting the warning. The case where the user moves away from the dangerous area is a case where the distance between the user position and the dangerous area position is equal to or greater than the distance threshold.

The processor 26 may temporarily stop outputting the warning. For example, the user can set whether to temporarily stop outputting the warning. In a case where the temporary stop is set by the user, the processor 26 temporarily stops outputting the warning. For example, the processor 26 does not output a warning.

Further, in a case where the user is approaching the dangerous area at a moving speed that is equal to or higher than a predetermined speed, the processor 26 may not output a warning. The case where the user is approaching the dangerous area at a moving speed that is equal to or higher than a predetermined speed is the case where the moving speed of the user is equal to or higher than the predetermined speed in a case where the distance between the user position and the dangerous area position is less than the distance threshold. In a case where the user is moving at a moving speed equal to or higher than a predetermined speed, the processor 26 does not output a warning because it is predicted that the user will immediately move away from the dangerous area even in a case where the user approaches the dangerous area. For example, in a case where the user moves by a car and the moving speed becomes equal to or higher than a predetermined speed, the processor 26 does not output a warning.

In a case where the danger occurring in the dangerous area is eliminated, the setting of the dangerous area may be canceled. In this case, the processor 26 does not output a warning even in a case where the user approaches an area where the dangerous area setting is cancelled.

The processor 26 may change the warning output mode according to the danger level of the dangerous area. For example, according to the danger level, the processor 26 may change the size and color of the warning text to be displayed, change the type of warning sound and the magnitude of warning sound, or change the magnitude of vibration of the information processing apparatus 10. Specifically, as the danger level becomes higher, the processor 26 may increase the warning text, change the color of the warning text to a conspicuous color (for example, red), increase the magnitude of warning sound, or increase the magnitude of the vibration of the information processing apparatus 10.

The processor 26 may acquire information related to the dangerous area and output a warning only in a specific area, and may not output the warning without acquiring information related to the dangerous area in areas other than the specific area. The specific area is, for example, an area where the frequency of occurrence of danger is high or an area where danger is predicted to occur.

The processor 26 may acquire only information related to a dangerous area where a specific danger has occurred, and may not acquire information related to a dangerous area where a danger other than the specific danger has occurred. The specific danger is, for example, a danger that is predicted to harm a user who passes through the dangerous area or a user who approaches the dangerous area, and is determined in advance.

The processor 26 may change the warning output mode according to the number of users approaching the dangerous area. The processor 26 acquires position information of each user from a terminal device (for example, a smartphone) carried by each user, and recognizes the position of each user. For example, the processor 26 changes the warning output mode, in a case where one user is approaching the dangerous area and in a case where a plurality of users are approaching the dangerous area. Specifically, since it is assumed that it is more dangerous for one person to approach the dangerous area than for a plurality of people to approach the dangerous area, in a case where one user is approaching the dangerous area, the processor 26 may increase the warning text to be displayed, change the color of the warning text to a conspicuous color (for example, red), increase the warning sound, or increase the magnitude of the vibration of the information processing apparatus 10, than in a case where a plurality of users is approaching the dangerous area. The processor 26 may change the warning output mode step by step according to the number of users approaching the dangerous area. In a case where the number of users less than the first threshold are approaching the dangerous area, a case where the number of users equal to or greater than the first threshold and less than the second threshold which is greater than the first threshold are approaching the dangerous area, and a case where the number of users equal to or greater than the second threshold are approaching the dangerous area, the processor 26 may change the warning output mode. For example, the processor 26 outputs a warning so as to be more conspicuous as the number of persons is smaller.

In Example 3 as well, the dangerous area may be updated at a predetermined time interval or may be updated according to a user's instruction.

### EXAMPLE 4

In Example 4, in a case of guiding a route from the departure place to the destination, the processor 26 guides the route to reach the destination by connecting the safety areas. The safety area is a position where the biological information representing the safety information described above is measured, or a predetermined non-warning area including the position. As another example, the safety area may be determined without being based on biological information. For example, the safety area may be determined by an administrative organization or a private organization. An area where crime prevention patrol is performed, an area where there are many street lamps, an area having a predetermined area including a position where a police station or police box is installed, and the like may be determined as a safety area. In this case, the processor 26 acquires information on the safety area from a device used in an administrative organization or a private organization through a communication path. The information related to the safety area includes, for example, position information indicating the position of the safety area, information indicating the reason why the safety area is safe (for example, patrol is performed, or there is a police station), and the like. The safety area may include a position where the biological information representing the safety information described above is measured, or a non-warning area including the position, and an area determined without being based on biological information.

The route to reach the destination by connecting the safety areas is the route that passes through the one safety area in a case where there is only one safety area, and the route that passes through a plurality of safety areas in a case where there is a plurality of safety areas. Safety areas may be areas separated from each other, or areas partially overlapping each other.

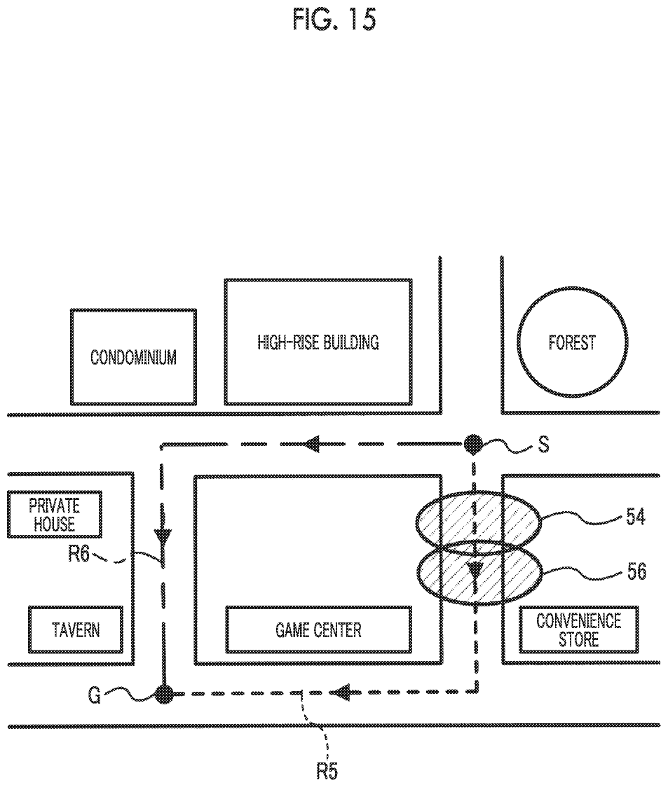

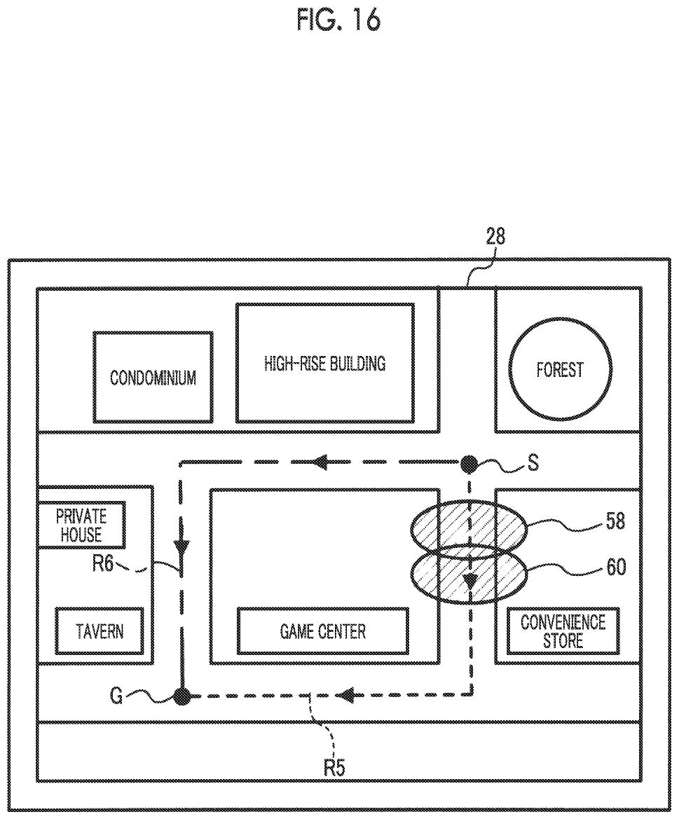

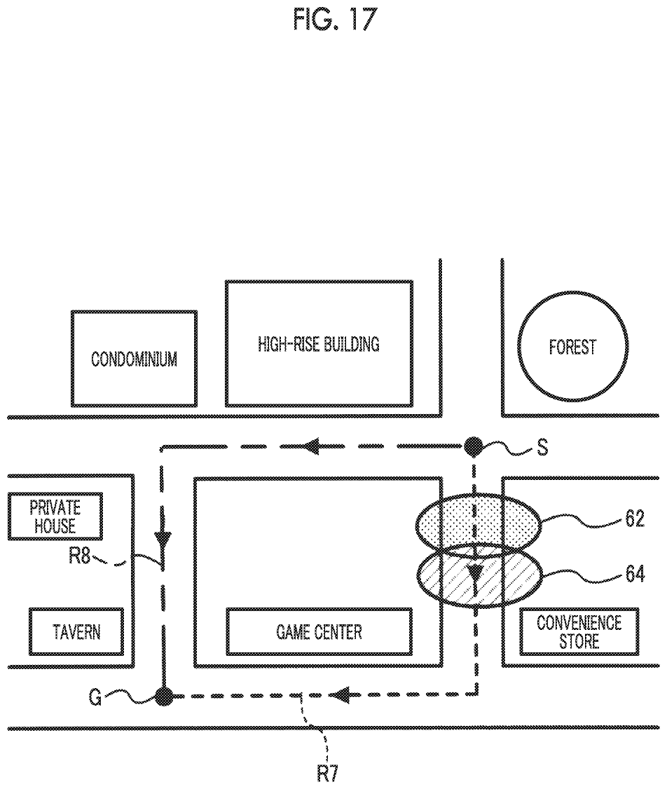

For example, as shown in FIG. 15, a departure place S and a destination G are set, and routes R5, R6 from the departure place S to the destination G are searched. The route R5 is a route that connects the safety areas 54 and 56 together. The safety area 54 is an area having a predetermined size including, for example, a position where a police station is installed. The safety area 56 is an area where crime prevention patrol is performed, for example. The route R6 is a route that does not pass through the safety area. In this case, the processor 26 guides the route R5 passing through the safety areas 54, 56 without guiding the route R6. In a case where other safety areas other than the safety areas 54, 56 exists, the processor 26 guides a route passing through the safety areas 54, 56 and other safety areas. As shown in FIG. 16, the map image 28 is displayed on the display unit of the UI 16, and the processor 26 guides the route R6. The processor 26 may display an image 58 representing the safety area 54 and an image 60 representing the safety area 56 on the map image 28.

Further, the processor 26 may display information indicating safe means of transportation on the display unit of the UI 16, or may emit a sound representing means of transportation from a speaker.

In a case where a plurality of routes passing through the safety area are searched, the processor 26 may prioritize each route and display each route on the display unit of the UI 16.

For example, the processor 26 determines the priority, based on the safety level of the safety area. For example, a danger level is determined in advance for each safety reason. For example, the safety level of the area including the position where the police station is installed is the highest, and the safety level of the area where the crime prevention patrol is performed is the second highest. Of course, these safety levels are only examples, and other safety levels may be set. The processor 26 determines the safety level of the safety area, based on the reason why the safety area is safe, included in the information related to the safety information, and determines the priority based on the safety level. A route passing through a safety area with a higher safety level has a higher priority.

Further, the processor 26 may determine the priority, based on the safety level, the required time, and the cost.

In addition, elements to be regarded as important may be set among a plurality of elements (for example, a safety level, required time, cost) for determining priorities. The setting may be performed by the user or may be automatically set by the processor 26. For example, in a case where the user sets the cost as the element to be regarded as important, the processor 26 gives higher priority to a route with a lower cost.

Further, the processor 26 may share a route that passes through the safety area with a plurality of other users. Other users are users other than the user who sets the route, and are designated by the user who sets the route. Other users may be designated in advance, or may be designated when the route is shared with other users. For example, the processor 26 may transmit information indicating the route to the addresses of other users by e-mail, or may make other users browse the route by a social media or social networking service (SNS). In a case where the information indicating the route is stored in an external device such as a server, the processor 26 may allow other users to access the information indicating the route stored in the external device.

In a case where there are a plurality of routes passing through the safety area, the processor 26 may share the plurality of routes with a plurality of other users. In this case, the processor 26 determines a route to be guided, based on an agreement among a plurality of users sharing the plurality of routes, from the plurality of routes. Then, the processor 26 guides the determined route. For example, each user who shares the plurality of routes designates a route to be guided using his or her terminal device (for example, a PC or a smartphone). Further, the user whose route is to be guided also designates the route to be guided using his or her information processing apparatus 10. Note that the number of routes that can be designated by the user may be one, or may be two or more. The number may be designated by the user whose route is to be guided. In a case where a route is designated by another user, information indicating the route designated by the other user is transmitted to the information processing apparatus 10 from the terminal device of the other user. For example, the processor 26 may determine the route with the highest number of designated routes as the routes to be guided, or determine one or a plurality of routes having the designated number equal to or greater than the threshold as the routes to be guided, or determine a route designated by all users as a route to be guided. In a case where a plurality of routes are determined as routes to be guided, a user whose route is guided designates a route to be finally guided from among the plurality of routes. A weight may be set for each user, and the weight may be reflected in a designated number. Further, in a case where another user (such as an administrator) who has an authority to set a route designates a route to be guided, the processor 26 may guide the route.

The safety area may be updated at a predetermined time interval or may be updated according to a user's instruction.

### EXAMPLE 5

In Example 5, in a case of guiding a route from the departure place to the destination, the processor 26 guides the route according to the priorities of the safety information and the dangerous area. The meaning of the dangerous area according to Example 5 is identical to the dangerous area according to Example 2. The meaning of the safety area according to Example is identical to the safety area according to Example 4.

For example, the processor 26 guides the route according to the priorities of the position where the biological information representing the safety information is measured and the position where the biological information representing the danger information is measured. For example, in a case where both the safety area and the dangerous area exist on the route from the departure place to the destination, the processor 26 determines the route, based on the priorities of the safety area and the dangerous area. The priority is determined based on, for example, the danger level and the safety level.

For example, in a case where both a safety area and a dangerous area exist on the route from the departure place to the destination, and the safety level of the safety area is higher than the danger level of the dangerous area, the processor 26 determines the route as a route to be guided, and guides the route. On the other hand, in a case where the safety level of the safety area is lower than the danger level of the dangerous area, the processor 26 determines another route as a route to be guided, and guides the other route.

Specifically, the processor 26 determines the priority, based on the safety contents of the safety area (that is, the reason why the safety area is safe), the contents of the incident or accident that has occurred in the dangerous area, the occurrence time of the incident or accident that has occurred in the dangerous area, the frequency of the incident or accident that has occurred in the dangerous area. For example, the safety level of the safety area is determined, based on the safety contents of the safety area. Further, the danger level of the dangerous area is determined based on the contents, occurrence time, and occurrence frequency of the incident or accident that has occurred in the dangerous area.

Further, the required time, cost, or the like from the departure place to the destination may be used as an element for determining the route to be guided. For example, the processor 26 may guide a route with a short required time or a route with low cost. For example, even in a case of the route passing through the dangerous area, the processor 26 may guide the route passing through the dangerous area, in a case where the required time is shorter than the route passing through the safety area or in a case where the cost is low.

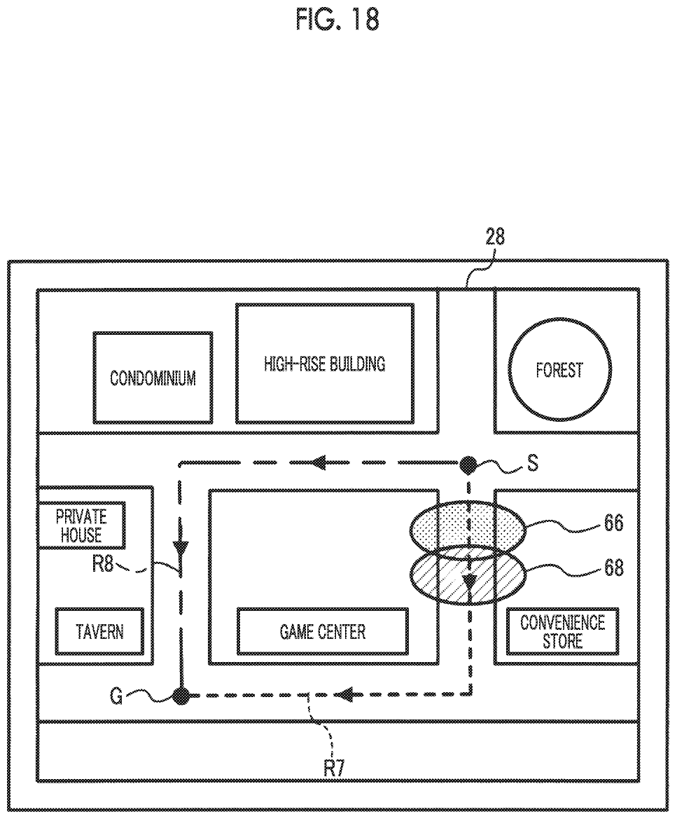

Further, in a case where the dangerous area and the safety area partially overlap, the processor 26 may guide a route that avoids the partially overlapping area. For example, as shown in FIG. 17, a departure place S and a destination G are set, and routes R7, R8 from the departure place S to the destination G are searched. The route R7 is a route that passes through the dangerous area 62 and the safety area 64. Further, the dangerous area 62 and the safety area 64 partially overlap. For example, in a case where an accident or incident occurs in the dangerous area 62 including a part of the safety area 64 where crime prevention patrol is performed, the dangerous area 62 and the safety area 64 partially overlap. The route R8 is a route that does not pass through the dangerous area and the safety area. In this case, the processor 26 guides a route that avoids an area where the dangerous area 62 and the safety area 64 overlap. That is, in a case where an accident or incident occurs even in the safety area 64, the processor 26 guides a route that avoids the area where the accident or incident has occurred. In the example shown in FIG. 17, the processor 26 guides the route R8. As shown in FIG. 18, the map image 28 is displayed on the display unit of the UI 16, and the processor 26 guides the route R8. The processor 26 may display an image 66 representing the dangerous area 62 and an image 68 representing the safety area 64 on the map image 28.

Further, the processor 26 may display information indicating safe means of transportation on the display unit of the UI 16, or may emit a sound representing means of transportation from the speaker.

In a case where navigation is performed in each of the above exemplary embodiments, the map image and the route to be guided may be displayed on a device other than the information processing apparatus 10. Danger information and safety information may also be displayed on a device other than the information processing apparatus 10. For example, a map image and a route to be guided may be displayed on a terminal device (for example, a smartphone) carried by the user when moving. The same applies to danger information and safety information. In this case, the information processing apparatus 10 may function as a device such as a server, and may transmit map image data and data of the route to be guided, to the terminal device to which the user to be guided has logged in. Of course, map image data is transmitted to a terminal device from a device other than the information processing apparatus 10 (for example, an external server), and data of the route to be guided, danger information, and safety information may be transmitted from the information processing apparatus 10 to the terminal device. The route may be guided by voice.

In the embodiments above, the term “processor” refers to hardware in a broad sense. Examples of the processor includes general processors (e.g., CPU: Central Processing Unit), dedicated processors (e.g., GPU: Graphics Processing Unit, ASIC: Application Integrated Circuit, FPGA: Field Programmable Gate Array, and programmable logic device). In the embodiments above, the term “processor” is broad enough to encompass one processor or plural processors in collaboration which are located physically apart from each other but may work cooperatively. The order of operations of the processor is not limited to one described in the embodiments above, and may be changed.

The foregoing description of the exemplary embodiments of the present invention has been provided for the purposes of illustration and description. It is not intended to be exhaustive or to limit the invention to the precise forms disclosed. Obviously, many modifications and variations will be apparent to practitioners skilled in the art. The embodiments were chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

What is claimed is:

1. A method comprising the steps of:
associating a user's attribution information, relief or relaxation information that is based on measured biological information, user location information, and information relating to the measured time by a processor; and
determining that the user is in a state of relief or relaxation in response to the value of the measured biological information becoming less than a predetermined value.

2. The method of claim 1, wherein:
the biological information is brain waves; and
the state of relaxation is determined by analyzing features of the measured values of brain waves.

3. The method of claim 2, wherein: the state of relaxation is determined when the alpha wave is dominant over other frequency bands of the brain waves among the measured values of brain waves.

4. The method of claim 1, wherein the relaxation information is obtained by using multiple types of biological information.

5. The method of claim 1, wherein artificial intelligence is utilized to obtain relaxation information from the biological information.

6. The method of claim 1, wherein information relating to a building located at the user's location is associated with the relaxation information.

7. The method of claim 1, wherein the relaxation information associated with the location information is sent to a social network service.

8. The method of claim 1, wherein the user is prompted to confirm whether or not the relaxation information should be recorded.

9. The method of claim 1, wherein information related to how to move when the biological information is being measured is associated.

10. The method of claim 1, wherein a recommended route or method of movement for the user to move to a specified destination while in a state of relaxation is provided.

11. The method of claim 1, wherein an area on a map containing multiple relaxation information sources is displayed.

12. A device comprising:
a processor configured to:
associate a user's attribution information, relief or relaxation information that is based on measured biological information, user location information, and information relating to the measured time; and
determine that the user is in a state of relief or relaxation in response to the value of the measured biological information becoming less than a predetermined value.

13. A non-transitory computer-readable recording medium having stored thereon instructions for causing a computer system to perform a method comprising the steps of:
associating a user's attribution information, relief or relaxation information that is based on measured biological information, user location information, and information relating to the measured time by a processor;
determining that the user is in a state of relief or relaxation in response to the value of the measured biological information becoming less than a predetermined value.
